(19) United States
(12) Patent Application Publication
(10) Pub. No.: US 2025/0345242 A1
(43) Pub. Date: Nov. 13, 2025

(54) VIAL ADAPTER AND FILL ROD FOR USER-WEARABLE INFUSION PUMP SYSTEM

(71) Applicant: Tandem Diabetes Care, INC, San Diego, CA (US)

(72) Inventors: Amy Turnlund, San Diego, CA (US); Bob Weiller, San Diego, CA (US); Byron Cohea, San Diego, CA (US); David Lin, San Diego, CA (US); Tinh Tran, San Diego, CA (US); Jacob Pearlman, San Diego, CA (US); Ed Ho, San Diego, CA (US); Steph Habif, San Diego, CA (US); Steve Vargus, San Diego, CA (US); Amrith Karunaratne, San Diego, CA (US); Dan Morrison, San Diego, CA (US)

(21) Appl. No.: 19/205,083

(22) Filed: May 12, 2025

Related U.S. Application Data
(60) Provisional application No. 63/645,517, filed on May 10, 2024.

(51) Int. Cl. A61J 1/14; A61J 1/20; A61M 5/14; A61M 5/142

(52) U.S. Cl. CPC A61J 1/1406; A61J 1/2006; A61J 1/2096; A61M 5/1413; A61M 5/14244; A61M 2005/1401

(57) ABSTRACT

Embodiments of the present disclosure relate to devices and methods for filling a medicament cartridge of a pump, including a user-wearable, ambulatory infusion pump. A vial adapter can be provided that can releasably secure a medicament cartridge and a vial of medicament. A fill rod can be releasably attached to a plunger of the medicament cartridge. A user can hold the vial adapter, with the medicament cartridge and vial secured thereto, in one hand and operate the fill rod with the other hand to fill the medicament cartridge with medicament from the vial.

### PRIORITY CLAIM

This application claims the benefit of U.S. Provisional Application No. 63/645,517 filed May 10, 2024, which is hereby incorporated by reference herein in its entirety.

### TECHNICAL FIELD

The present disclosure relates to medical pumps for delivering medicament to a patient and, more specifically, to a user-wearable pump.

### BACKGROUND

There are many applications in academic, industrial, and medical fields that benefit from devices and methods that are capable of accurately and controllably delivering fluids, such as liquids and gases, that have a beneficial effect when administered in known and controlled quantities. Such devices and methods can be particularly useful in the medical field where treatments for many patients include the administration of a known amount of a substance at predetermined intervals.

One category of devices for delivering such fluids is that of pumps that have been developed for the administration of insulin and other medicaments for those suffering from both type I and type II diabetes. Some pumps configured as portable infusion devices can provide continuous subcutaneous medicament injection and/or infusion therapy for the treatment of diabetes. Such therapy may include, e.g., the regular and/or continuous injection or infusion of insulin into a person suffering from diabetes and offer an alternative to multiple daily injections of insulin by an insulin syringe or an insulin pen. Such pumps can be ambulatory/portable infusion pumps that are worn by the user and may use replaceable cartridges. Such pumps can deliver medicaments other than or in addition to insulin, such as glucagon. Examples of such pumps and various features that can be associated with such pumps include those disclosed in U.S. Pat. No. 8,287,495, which is hereby incorporated herein by reference in its entirety.

One type of pump that has been developed is a patch pump, or micro pump. Patch pumps generally are small pumps that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Not unlike other types of pumps, but perhaps more typically, patch pumps can be at least in part disposable, meant to be worn for a period of time such as, e.g., a day or two, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament. Still other patch pumps may include a rechargeable durable component as well as a refillable and reusable medicament cartridge.

An insulin pump user's first interaction with a patch pump is typically to fill the cartridge with insulin from an insulin vial. This process may use a vial adapter to retain the insulin vial and a fill rod to aid in transferring from the vial into the cartridge. The amount of physical strength, dexterity, vision, and health education needed to fill a patch pump cartridge varies across pump user groups. In the event that these user needs are not tended to, the result may be a cartridge that is too difficult to fill, an unusable cartridge, wasted insulin, and/or disruption or attrition from insulin pump therapy. As such, a design should be optimized to meet the needs of as many patch pump users as possible resulting in a successful cartridge fill and load.

### SUMMARY

Embodiments of the present disclosure relate to devices and methods for filling a medicament cartridge of a pump, including a user-wearable, ambulatory infusion pump. A vial adapter can be provided that can releasably secure a medicament cartridge and a vial of medicament. A fill rod can be releasably attached to a plunger of the medicament cartridge. A user can hold the vial adapter, with the medicament cartridge and vial secured thereto, in one hand and operate the fill rod with the other hand to fill the medicament cartridge with medicament from the vial.

In an embodiment, a vial adapter for a user-wearable infusion pump system can include a cartridge interface including a locking projection configured to releasably lock a medicament cartridge of a user-wearable infusion pump onto the cartridge interface and a vial interface configured to releasably secure a medicament vial. A septum can provide a fluid communication path between a medicament vial secured in the vial interface and a medicament cartridge locked onto the cartridge interface.

In embodiments, an ambulatory infusion pump system can include an ambulatory infusion pump including a refillable medicament cartridge and a vial adapter configured to facilitate filling of the medicament cartridge with a medicament from a medicament vial. The vial adapter can include a cartridge interface including a locking projection configured to releasably lock the medicament cartridge onto the cartridge interface, a vial interface configured to releasably secure the medicament vial and a septum configured to provide a fluid communication path between the medicament vial and the medicament cartridge.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict a patch pump system according to an embodiment of the disclosure.

FIGS. 2A-2B depicts the patch pump system of FIGS. 1A-1B.

FIGS. 3A-3D depict a vial adapter that can be used with the patch pump system of FIGS. 1A-1B according to an embodiment of the disclosure.

FIGS. 4A-4B depict a portion of the vial adapter of FIGS. 3A-3C with a vial inserted therein according to an embodiment of the disclosure.

FIG. 5 depicts a fill rod for use with the vial adapter of FIGS. 4A-4B.

FIGS. 6A-6B depicts an assembly of the cartridge of the patch pump system of FIGS. 1A-1B inserted onto the vial adapter of FIGS. 3A-3C and including the fill rod of FIG. 5.

FIGS. 7A-7C depict the assembly of FIG. 6 during a cartridge filling procedure.

FIGS. 8A-8D depict a vial adapter that can be used with the patch pump system of FIGS. 1A-1B according to an embodiment of the disclosure.

FIG. 9 depicts a fill rod for use with the vial adapter of FIGS. 8A-8D.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

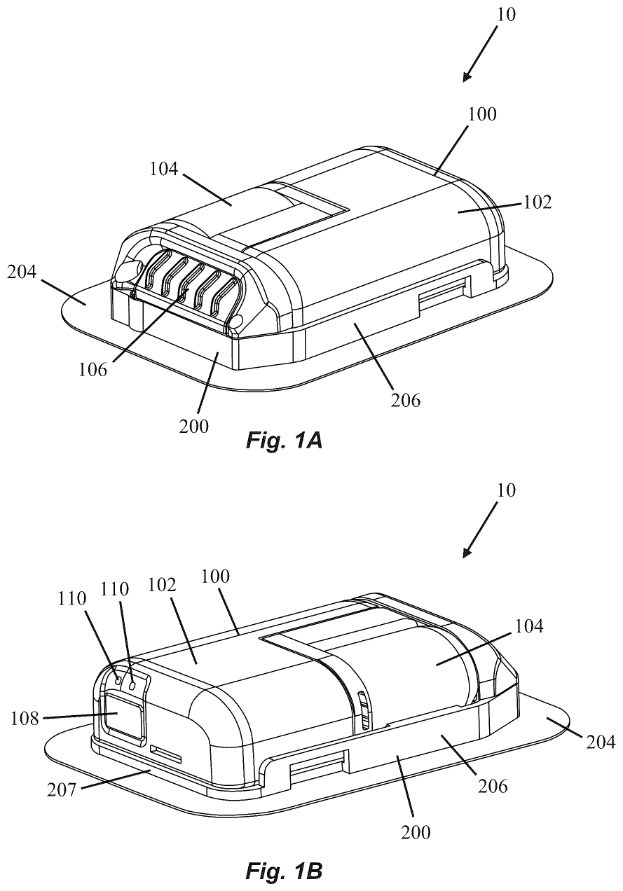

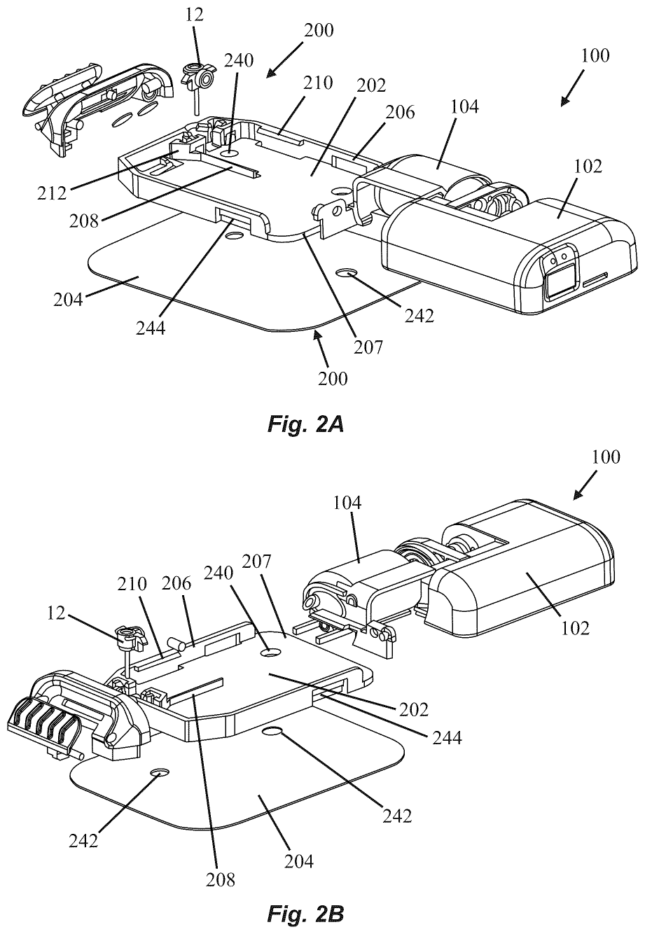

FIGS. 1A-1B and 2A-2B depict an ambulatory infusion pump system 10 according to an embodiment of the disclosure. System 10 can include an infusion pump 100 and a pump holder or tray 200 that can be used to releasably contain infusion pump 100 and that can be worn on a body of a user.

Pump 100 can include a drive unit 102 including a drive mechanism that mates with a recess in a medicament cartridge 104 to releasably hold the cartridge 104 on the drive unit 102. Further details regarding embodiments of a drive mechanism for delivering a medicament such as insulin from the medicament cartridge to a user and for attaching the cartridge to the drive unit can be found in U.S. Pat. Nos. 10,279,107 and 9,993,595, each of which is incorporated herein by reference in its entirety. In some embodiments, drive unit 102 is a durable component having a rechargeable battery and cartridge 104 is disposable and intended to be disposed of after the medicament in cartridge has been used. In other embodiments, cartridge can be refillable such that both components are durable. Pump 102 can further include a switch 106 configured to releasably lock the pump 102 onto the pump holder 200. Further details regarding such a pump can be found in U.S. Patent Publication No. U.S. 2024/0226423, which is hereby incorporated by reference herein in its entirety.

In embodiments, pump 100 can interface with a cannula 12 that extends through the pump holder 200 to deliver medicament to an infusion site on the user's body directly beneath the pump holder 200. In other embodiments, pump 100 can interface with infusion tubing to deliver medicament from the pump through the tubing to an infusion site displaced from the pump holder 200. In some embodiments, the pump 100 can be configured to interchangeably be used with a cannula directly beneath the pump holder 200 or an infusion site displaced from the holder 200 via infusion tubing. In some of those embodiments, drive unit 102 can be configured to attach to alternative cartridge types, with a first cartridge type including infusion tubing and a second cartridge type configured to deliver through a cannula extending directly beneath the pump 100. In various embodiments, pump 100 can be disconnected from a user's body and reattached using the same infusion site to enable the user to remove the pump for a limited period of time for certain activities, such as, e.g., charging the battery, taking a shower, playing sports, etc. without having to insert a cannula at a new infusion site.

In embodiments, pump includes a processor that controls the operations of the pump and may communicate in either one-way or two-way modes to, e.g., receive operational commands and/or other signals, including data, from a separate device and/or, e.g., to send signals, including data to a separate device. Pump can include one or more buttons configured to cause the processor to initiate one or more functions. In the depicted embodiment, pump 100 includes only a single button 108, although more than one button may be present on pump 100. Button 108 can be configured to, for example, initiate delivery of medicament (e.g., a “quick bolus”). Any single button such as button 108 can be utilized to execute a plurality of functions or operations. For example, a single press of button may initiate one function, holding the button down for a predetermined period of time may initiate another function, etc. Because the depicted pump 100 optionally does not itself include a display or user interface, information and feedback regarding medicament delivery or dosing or other functions initiated with button 108 can be communicated to and displayed on a remote control device or other device having a display and/or other type of user interface.

In embodiments, pump 100 includes a light source, such as a light emitting diode (LED) 110. Light source 110 can be configured to provide user feedback regarding user input and/or the performance of a desired function. For example, in one embodiment, light source 110 can illuminate or blink one or more times to indicate that the one or more buttons 108 have been activated and/or that a desired function has been initiated. In one embodiment, pump 100 can additionally and/or alternatively vibrate and/or provide audible notifications to indicate that the one or more buttons 108 have been activated and/or that a desired function has been initiated or, e.g., to provide user feedback regarding user input and/or the performance of the desired function. Illumination of light source 110 and/or vibrations and/or audible notifications may be executed in any number of patterns, frequencies, durations, sequences, combinations, colors, brightness levels, etc. to indicate particular information, such as particular input received and/or particular functions or operations enabled and/or initiated, to the pump user or caregiver. In the depicted embodiment, the pump 100 includes two indicator lights 110.

Pump holder 200 can include a tray body 202 having a shape generally matching the shape of pump 100 and an adhesive patch 204 configured to releasably retain the pump holder 200 and pump 100 on the user's body. One or more perimeter walls 206 can extend around at least a portion of the perimeter of the tray body 202 to aid in retaining and guiding the pump 100 onto the holder 200. In the depicted embodiment, tray body 202 can include one end 207 that does not include a perimeter wall to aid in sliding pump 102 onto tray body 202. Pump holder 200 can include various other features that aid in retaining and/or guiding the pump onto holder 200, including a guide rail 208, retention projection 210 and a locking ramp 212. Pump holder 200 and patch 204 can include aligned apertures 240, 242 and retention walls 206 can include slots 244 that aid in temporarily retaining pump holder 200 in and releasing pump holder 200 from an insertion or applicator device configured to apply the pump holder 200 onto the user's skin.

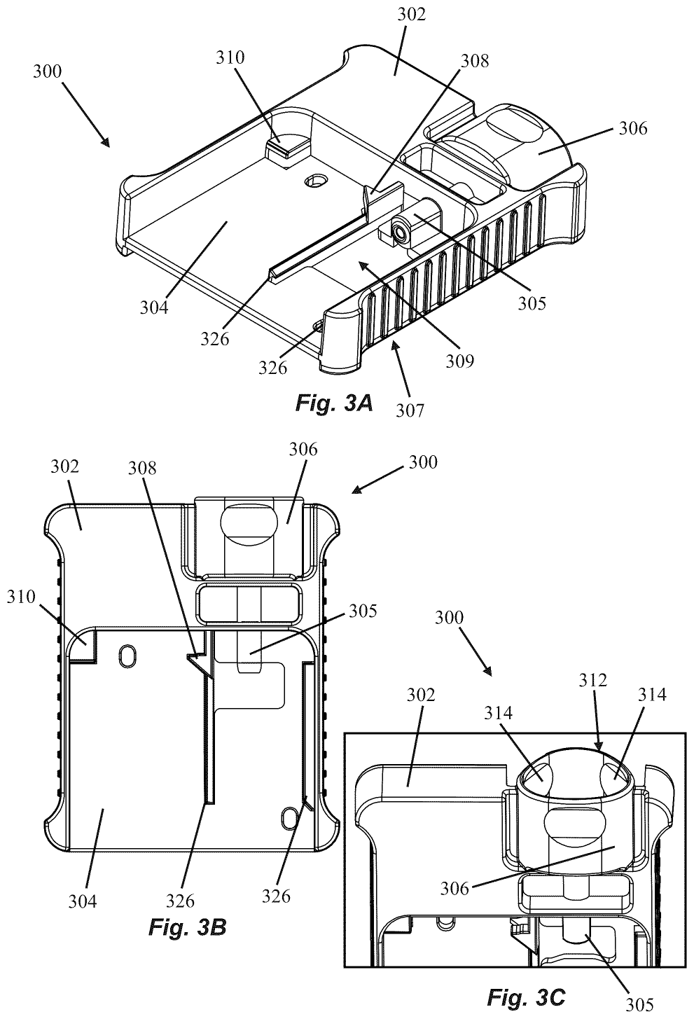

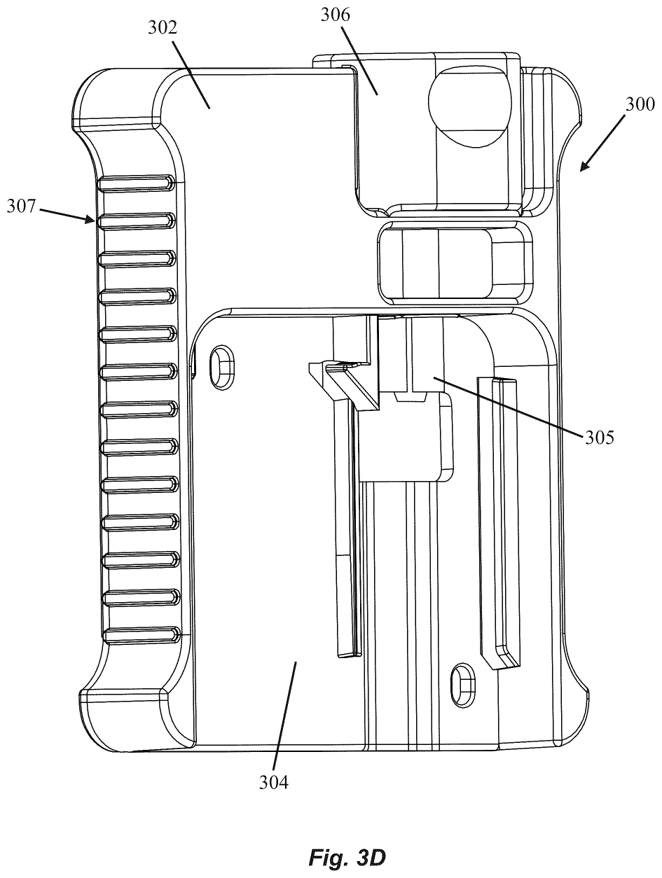

FIGS. 3A-3D depict a vial adapter 300 that can be used with the patch pump system of FIGS. 1A-1B according to an embodiment of the disclosure. Vial adapter 300 can include an adapter body 302. In embodiments, adapter body 302 can be comprised of a rigid plastic material. Vial adapter 300 can define a cartridge interface 304 designed to hold a medicament cartridge during a cartridge filling process and a vial interface 306 designed to hold a vial of medicament during the filling process. A needle interface 305 can contain a septum that facilitates a fluid connection between a cartridge and a vial, as will be described in more detail below. Adapter body 302 can further include a plurality of ribs 307 that aid a user in gripping vial adapter during a handheld filling procedure. Adapter body 302 may further include an aperture 309 that enables a cartridge inserted thereon to be viewed from the bottom side of vial adapter 300.

Cartridge interface 304 can include one or more guide rails 326 that serve to guide and retain a cartridge onto the vial adapter and to releasably lock the cartridge onto the vial adapter with locking projection 308. The same features of the pump 100 that interface with the pump holder 200 can interface with these features of the vial adapter 300. In some embodiments, pump 100 can automatically lock onto vial adapter 300 and can be removed from vial adapter 300 by sliding switch 106 to disengage a feature of pump 100 from the locking projection 308 to enable the pump 100 to be slid back off of the vial adapter 300. Cartridge interface 304 can also include an alignment projection 310 that interfaces with cartridge to aid in ensuring proper alignment of the cartridge on the cartridge interface 304.

Vial interface 306 can include a cavity 312 into which an end of a vial can be inserted. Retention protrusions 314 can extend inwardly into cavity 312. In operation, a glass lip of a vial can be pushed passed the retention protrusions 314 with the protrusions 314 then preventing the vial from inadvertently being removed from the cavity 312 during normal filling operations. Sufficient force can be applied following the filling procedure to pull the vial passed the retention protrusions 314 to remove the vial.

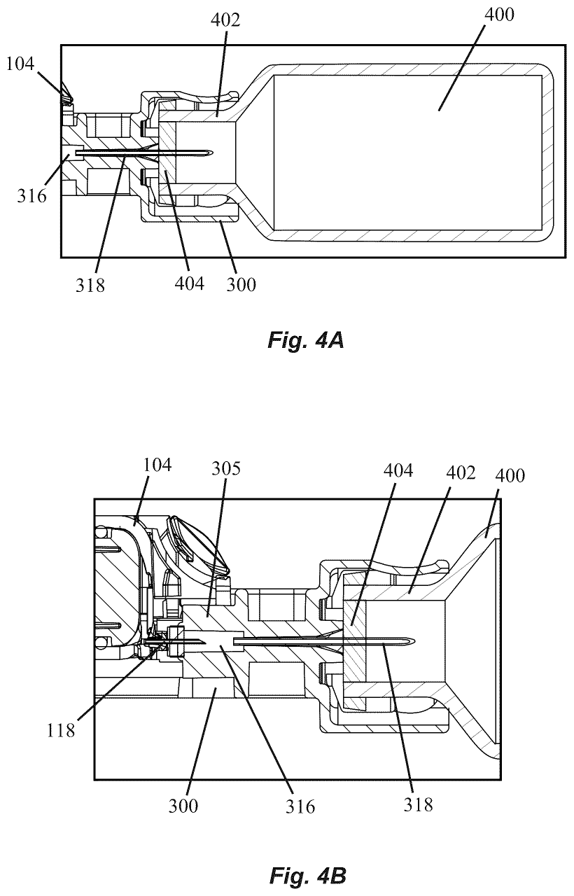

FIGS. 4A-4B depict a portion of vial adapter 300 with a vial 400 inserted therein according to an embodiment of the disclosure in order to illustrate the fluid connection between vial 400 and cartridge 104 facilitated by vial adapter. A septum 316 can be disposed within needle interface of vial adapter 300 and include a first needle 318 extending from septum 316 into cavity 312. When the vial head 402 is inserted into cavity 312, the first needle 318 pierces a membrane 404 covering the vial head 402 to put the septum 316 in fluid communication with the liquid contained in the vial 400. When the cartridge 104 is slid onto the vial adapter 300, a second needle 118 that extends from the cartridge 104 pierces the other end of the septum 316 to put the internal reservoir of the cartridge 104 in fluid communication with the septum. In this manner, a fluid path is created between the vial 400 and the cartridge 104 via the septum 316 contained in the vial adapter 300.

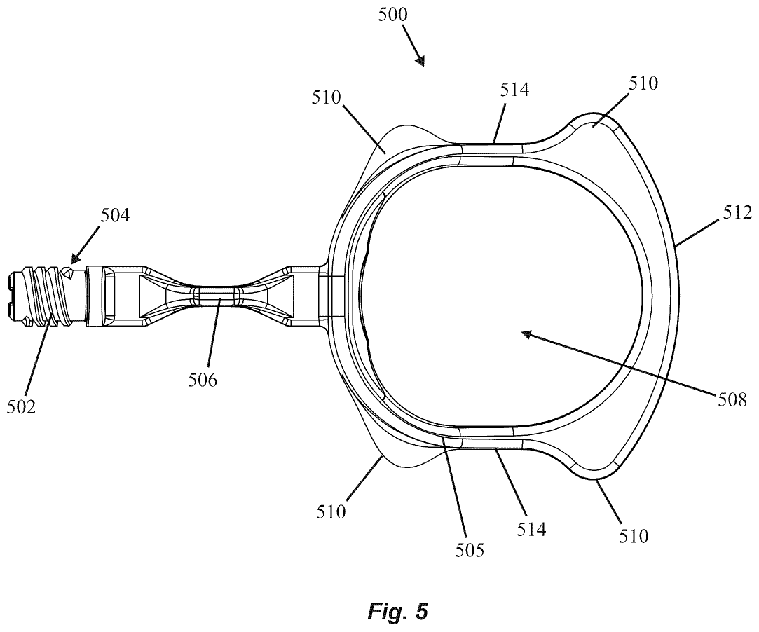

FIG. 5 depicts a fill rod 500 for use with the cartridge 104 and vial adapter 300 to fill the cartridge 104 with medicament from the vial 400. Fill rod 500 includes a connector 502 configured to be attached to a plunger within medicament reservoir. In the depicted embodiments, connector 502 includes threads 504 enabling the connector 502 to be rotated to be screwed into corresponding threads in the plunger. This connection prevents the fill rod from accidentally being disconnected during use. However, other means of connecting the fill rod 500 to the plunger are possible. Connector 502 can be attached to a handle 505 with a chain link connector 506. The chain link connector 506 provides flexibility to the fill rod 500 during translation of the fill rod 500 back and forth, while providing stiffness if the handle 505 is twisted. The flexibility during filling minimizes the amount of force that can be applied to the O-rings on the plunger to provide a better seal during filling and also reduces the possibility of the plunger tilting out of axis when pulling/pushing in a skewed direction from the plunger travel. The torsional strength to resist twisting enables substantial torque to be applied to reduce the likelihood of the fill rod 500 becoming loose. Handle 505 can be ergonomically designed to comprise a loop 508 and one or more protruding horns 510 that form a curved grip 512 and recesses 514 that aid a user in grasping and manipulating handle 504.

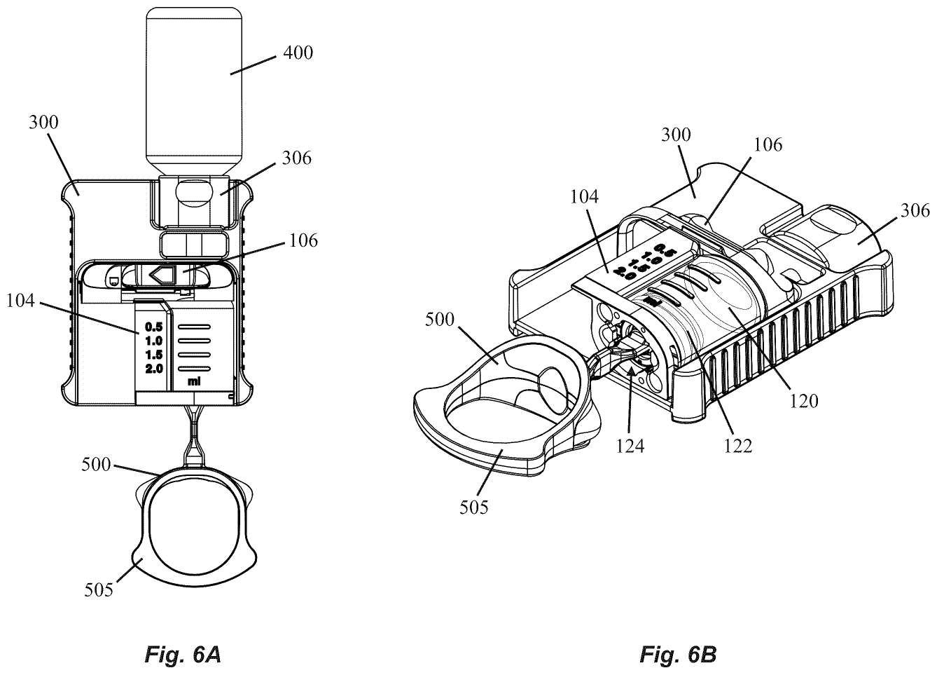

FIGS. 6A-6B depicts an assembly of a cartridge 104, a vial 400 and a fill rod 500 on vial adapter 300. Vial 400 (only depicted in FIG. 6A) has been inserted into vial interface 306 and fluid communication between vial 400 and septum 316 established via needle 318 as depicted in FIGS. 4A-4B. Cartridge 104 is releasably locked onto vial adapter 400 via locking projection 308 and switch 106 with internal reservoir 120 in fluid communication with septum 316 via needle 118 as also depicted in FIGS. 4A-4B. Fill rod 500 has been connected to plunger 122 in reservoir 120 through an open end 124 in cartridge 104.

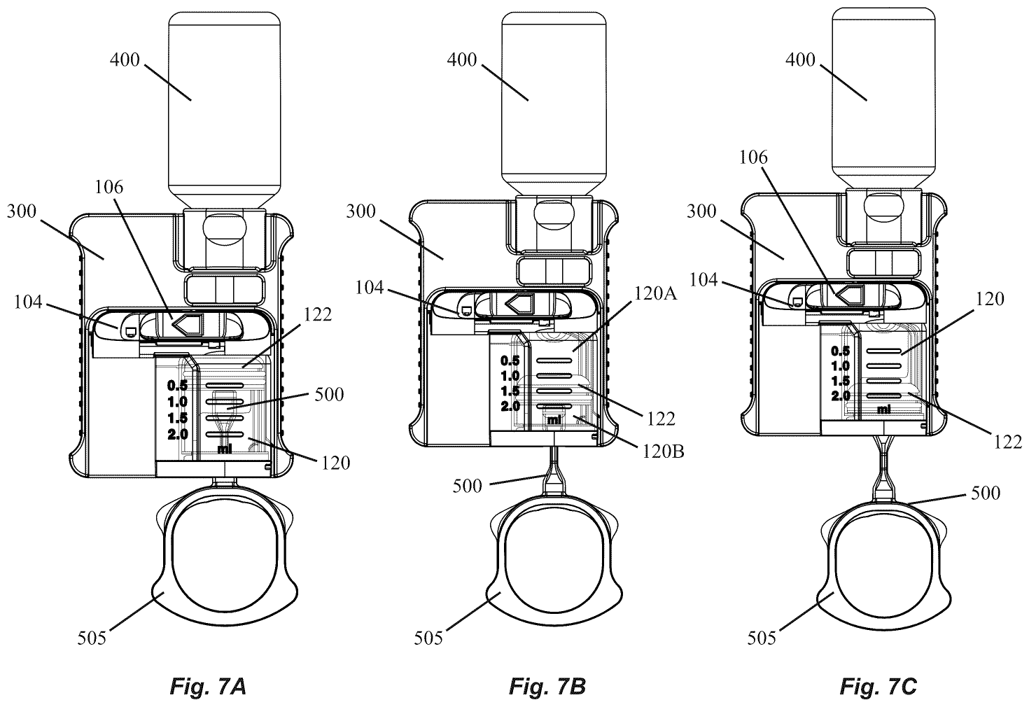

FIG. 7A-7C depict the assembly of FIG. 6A during a cartridge filling procedure. Vial adapter 300 is configured to enable a handheld filling procedure because the cartridge 104 is secured to the vial adapter 300 with locking projection 308 and the vial 400 is securely retained within the vial interface 306 by the retention protrusions 314 such that the user does not need to hold either component during filling. A user can therefore grasp the vial adapter 300 via the ribs 307 on the adapter body 302 with one hand and the handle 505 of the fill rod 500 with the other hand.

FIG. 7A depicts a cartridge 104 at the beginning of a fill procedure with an empty reservoir 120. The plunger 122 in the medicament reservoir 120 of cartridge is at a distal-most end of the reservoir relative to the user. The user can fill the reservoir 120 with medicament from the vial 400 by pulling on the fill rod 500 with handle 505 to draw medicament from the vial 400 through the septum 316 in the vial adapter 300 and into the reservoir 120. To aid in this process, the vial 400 may first be pressurized with air to enable the medicament to be more easily drawn out of the vial 400 and into the reservoir 120. The user may need to move the fill rod 500 back and forth during the filling procedure to remove bubbles from the liquid medication. FIG. 7B depicts a partially completed fill procedure in which the area 120A of the reservoir 120 distal of the plunger 122 is filled with medicament and the area 120B proximal of the plunger is still empty. In FIG. 7C, the fill procedure has been completed. The plunger 122 is at the proximal-most end of the reservoir 120 such that the reservoir is filled to 100% capacity with the medicament. In embodiments, reservoir 120 can comprise a transparent material and/or include graduated markings to aid a user in tracking the fill process and to enable to user to detect and remove any bubbles in the medicament. Following the filling procedure, the user can remove the fill rod 500 from the cartridge 104 by unscrewing or otherwise detaching the fill rod, the cartridge 104 from the vial adapter 300 by actuating the switch 106 and sliding the cartridge back off of the vial adapter and the vial 400 from the vial interface 306 by pulling the vial 400 with sufficient force to overcome the retention protrusions 314.

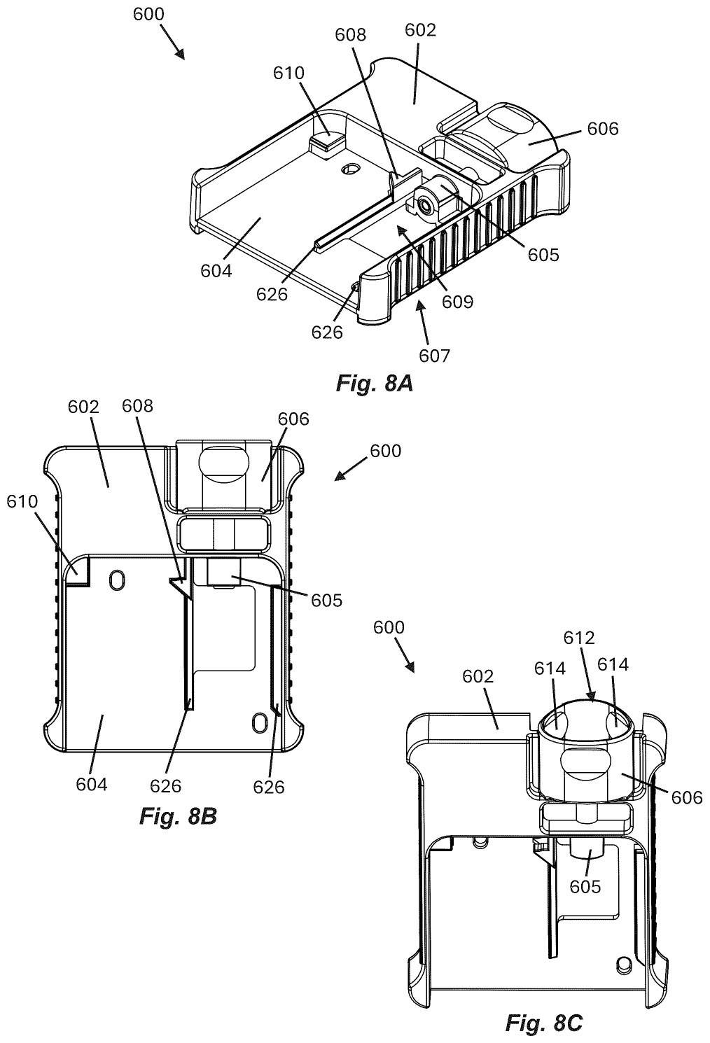

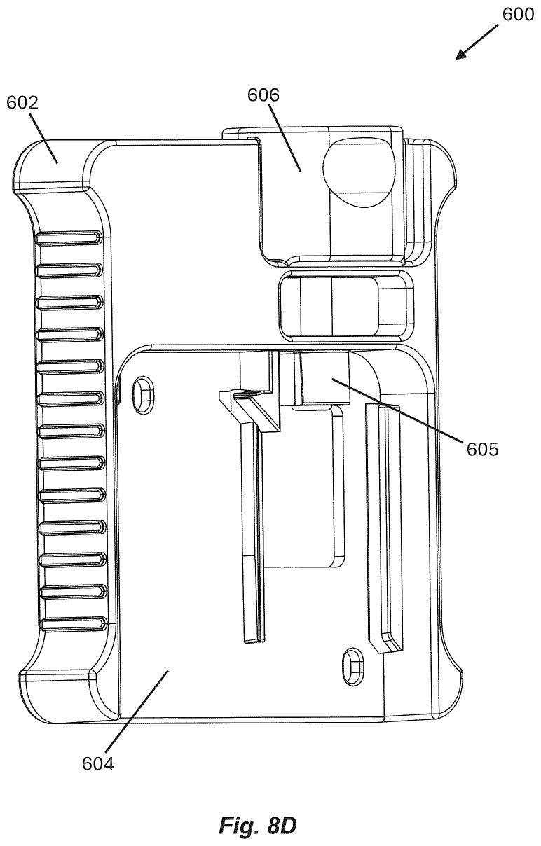

FIGS. 8A-8D depict another vial adapter 600 that can be used with a patch pump system such as the patch pump system of FIGS. 1A-1B according to an embodiment of the disclosure. Vial adapter 600 can include an adapter body 602. In embodiments, adapter body 602 can be comprised of a rigid plastic material. Vial adapter 600 can define a cartridge interface 604 designed to hold a medicament cartridge during a cartridge filling process and a vial interface 606 designed to hold a vial of medicament during the filling process. A needle interface 605 can contain a septum that facilitates a fluid connection between a cartridge and a vial. In this embodiment, needle interface 605 is shorter and wider than needle interface 305 of vial adapter 300, but the functionality remains the same. Adapter body 602 can further include a plurality of ribs 607 that aid a user in gripping vial adapter during a handheld filling procedure. Adapter body 602 may further include an aperture 609 that enables a cartridge inserted thereon to be viewed from the bottom side of vial adapter 600.

Cartridge interface 604 can include one or more guide rails 626 that serve to guide and retain a cartridge onto the vial adapter and to releasably lock the cartridge onto the vial adapter with locking projection 608. The same features of the pump 100 that interface with the pump holder 200 can interface with these features of the vial adapter 600. In some embodiments, pump 100 can automatically lock onto vial adapter 600 and can be removed from vial adapter 600 by sliding switch 106 to disengage a feature of pump 100 from the locking projection 608 to enable the pump 100 to be slid back off of the vial adapter 600. Cartridge interface 604 can also include an alignment projection 610 that interfaces with cartridge to aid in ensuring proper alignment of the cartridge on the cartridge interface 604.

Vial interface 606 can include a cavity 612 into which an end of a vial can be inserted. Retention protrusions 614 can extend inwardly into cavity 612. In operation, a glass lip of a vial can be pushed passed the retention protrusions 614 with the protrusions 614 then preventing the vial from inadvertently being removed from the cavity 612 during normal filling operations. Sufficient force can be applied following the filling procedure to pull the vial passed the retention protrusions 614 to remove the vial.

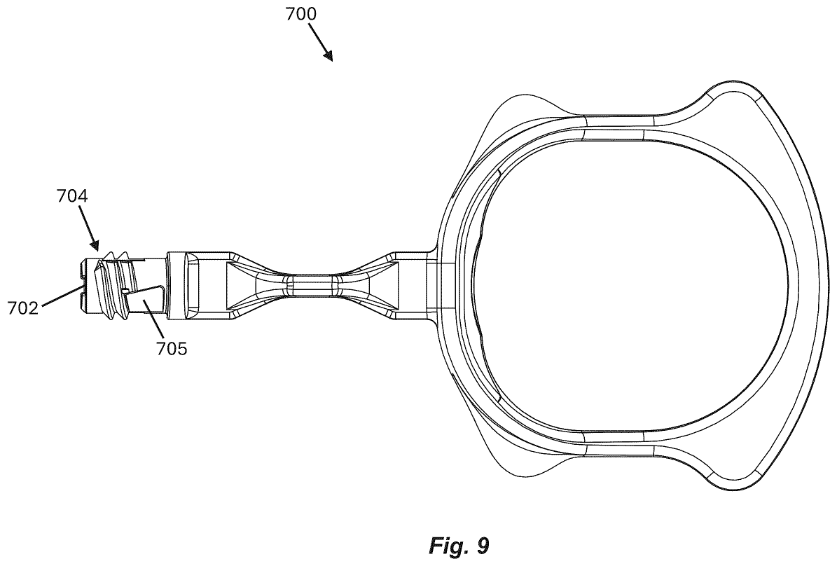

FIG. 9 depicts a fill rod 700 for use with the cartridge 104 and vial adapter 300 or 600 to fill the cartridge 104 with medicament from the vial 400. Fill rod 700 includes substantially the same components as fill rod 500 described above with respect to FIG. 5. In this embodiment, there is a locking recess 705 located at the termination of the threading 704 on connector 702 of fill rod 700. This recess 705 interlocks with the plunger 122 within the reservoir 120 of the cartridge 104 when the fill rod 700 is attached thereto (see FIGS. 6A-6B) to inhibit accidental disengagement of the fill rod 700 from the cartridge 104.

In embodiments, a vial adapter for a user-wearable infusion pump system can include a cartridge interface including a locking projection configured to releasably lock a medicament cartridge of a user-wearable infusion pump onto the cartridge interface and a vial interface configured to releasably secure a medicament vial. A septum can provide a fluid communication path between a medicament vial secured in the vial interface and a medicament cartridge locked onto the cartridge interface.

In an embodiment, the cartridge interface further includes one or more guide rails configured to guide the medicament cartridge to interlock with the locking projection.

In an embodiment, the locking projection is configured to automatically interlock with the medicament cartridge when the medicament cartridge is slid onto the cartridge interface.

In an embodiment, the vial interface comprises a cavity configured to receive an end of the medicament vial.

In an embodiment, the vial interface includes retention protrusions extending inwardly into the cavity to aid in securing the medicament vial in the vial interface.

In an embodiment, the vial adapter further comprises a needle interface containing the septum.

In an embodiment, the vial adapter further comprises a needle extending from the septum into the vial interface, the needle configured to pierce the medicament vial when the medicament vial is secured in the vial interface.

In an embodiment, the septum is configured to be pierced by a needle extending from the medicament cartridge when the medicament cartridge is locked onto the cartridge interface.

In an embodiment, the vial adapter further comprises a fill rod including a connector configured to be attached to a plunger within a medicament reservoir of the medicament cartridge when the medicament cartridge is locked onto the cartridge interface.

In an embodiment, the connector of the fill rod is configured to be threadedly attached to the plunger.

In an embodiment, the fill rod further comprises a handle and a chain-like connector connecting the handle to the connector.

In an embodiment, when the medicament vial is secured in the vial interface and the medicament cartridge is locked onto the cartridge interface to provide a fluid communication path therebetween via the septum, the medicament reservoir can be filled with medicament from the vial adapter by pulling the fill rod connected to the plunger away from the medicament vial. In an embodiment, the vial adapter comprises an adapter body defining the cartridge interface and the vial interface.

In an embodiment, the adapter body comprises a rigid plastic material.

In an embodiment, the adapter body comprises a plurality of ribs on an outer perimeter of the adapter body to aid a user in gripping the adapter body during a filling procedure.

In an embodiment, the adapter body comprises an aperture therethrough, the aperture positioned such that the medicament cartridge can be viewed through the aperture from a bottom side of the adapter body when the medicament cartridge is locked onto the cartridge interface.

In embodiments, an ambulatory infusion pump system can include an ambulatory infusion pump including a refillable medicament cartridge and a vial adapter configured to facilitate filling of the medicament cartridge with a medicament from a medicament vial. The vial adapter can include a cartridge interface including a locking projection configured to releasably lock the medicament cartridge onto the cartridge interface, a vial interface configured to releasably secure the medicament vial and a septum configured to provide a fluid communication path between the medicament vial and the medicament cartridge.

In an embodiment, the system further comprises a fill rod configured to be selectively attached to a plunger in the medicament cartridge, the fill rod configured to be pulled away from the medicament vial to draw the medicament from the medicament vial into the medicament cartridge.

In an embodiment, the medicament cartridge is configured to automatically lock onto the locking projection when the medicament cartridge is slid onto the cartridge interface.

In an embodiment, the system further comprises a slidable switch disposed on the medicament cartridge and wherein sliding the switch unlocks the medicament cartridge from the locking projection.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; and 12,214,159 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. patent application Ser. Nos. 17/368,968; 17/896,492; 18/398,543; 18/962,169; 19/003,140; 19/003,164 and 19/119,554.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

1. A vial adapter for a user-wearable infusion pump system, comprising:
a cartridge interface including a locking projection configured to releasably lock a medicament cartridge of a user-wearable infusion pump onto the cartridge interface;
a vial interface configured to releasably secure a medicament vial; and
a septum configured to provide a fluid communication path between a medicament vial secured in the vial interface and a medicament cartridge locked onto the cartridge interface.

2. The vial adapter of claim 1, wherein the cartridge interface further includes one or more guide rails configured to guide the medicament cartridge to interlock with the locking projection.

3. The vial adapter of claim 1, wherein the locking projection is configured to automatically interlock with the medicament cartridge when the medicament cartridge is slid onto the cartridge interface.

4. The vial adapter of claim 1, wherein the vial interface comprises a cavity configured to receive an end of the medicament vial.

5. The vial adapter of claim 4, wherein the vial interface includes retention protrusions extending inwardly into the cavity to aid in securing the medicament vial in the vial interface.

6. The vial adapter of claim 1, further comprising a needle interface containing the septum.

7. The vial adapter of claim 6, further comprising a needle extending from the septum into the vial interface, the needle configured to pierce the medicament vial when the medicament vial is secured in the vial interface.

8. The vial adapter of claim 6, wherein the septum is configured to be pierced by a needle extending from the medicament cartridge when the medicament cartridge is locked onto the cartridge interface.

9. The vial adapter of claim 1, further comprising a fill rod including a connector configured to be attached to a plunger within a medicament reservoir of the medicament cartridge when the medicament cartridge is locked onto the cartridge interface.

10. The vial adapter of claim 9, wherein the connector of the fill rod is configured to be threadedly attached to the plunger.

11. The vial adapter of claim 9, wherein the fill rod further comprises a handle and a chain-link connector connecting the handle to the connector.

12. The vial adapter of claim 9, wherein when the medicament vial is secured in the vial interface and the medicament cartridge is locked onto the cartridge interface to provide a fluid communication path therebetween via the septum, the medicament reservoir can be filled with medicament from the vial adapter by pulling the fill rod connected to the plunger away from the medicament vial.

13. The vial adapter of claim 1, wherein the vial adapter comprises an adapter body defining the cartridge interface and the vial interface.

14. The vial adapter of claim 13, wherein the adapter body comprises a rigid plastic material.

15. The vial adapter of claim 13, wherein the adapter body comprises a plurality of ribs on an outer perimeter of the adapter body to aid a user in gripping the adapter body during a filling procedure.

16. The vial adapter of claim 13, wherein the adapter body comprises an aperture therethrough, the aperture positioned such that the medicament cartridge can be viewed through the aperture from a bottom side of the adapter body when the medicament cartridge is locked onto the cartridge interface.

17. An ambulatory infusion pump system, comprising:
an ambulatory infusion pump including a refillable medicament cartridge; and
a vial adapter configured to facilitate filling of the medicament cartridge with a medicament from a medicament vial, the vial adapter including—
a cartridge interface including a locking projection configured to releasably lock the medicament cartridge onto the cartridge interface;
a vial interface configured to releasably secure the medicament vial; and
a septum configured to provide a fluid communication path between the medicament vial and the medicament cartridge.

18. The ambulatory infusion pump system of claim 17, further comprising a fill rod configured to be selectively attached to a plunger in the medicament cartridge, the fill rod configured to be pulled away from the medicament vial to drawing the medicament from the medicament vial into the medicament cartridge.

19. The ambulatory infusion pump system of claim 17, wherein the medicament cartridge is configured to automatically lock onto the locking projection when the medicament cartridge is slid onto the cartridge interface.

20. The ambulatory infusion pump system of claim 19, further comprising a slidable switch disposed on the medicament cartridge and wherein sliding the switch unlocks the medicament cartridge from the locking projection.
